(19) United States
(12) Patent Application Publication
(10) Pub. No.: US 2013/0034513 A1
(43) Pub. Date: Feb. 7, 2013

(54) USE OF A COSMETIC COMPOSITION COMPRISING AT LEAST ONE FILM-FORMING ELASTOMERIC POLYMER FOR TREATING HUMAN PERSPIRATION

(71) Applicant: Henri Samain, Bievres (FR)

(72) Inventor: Henri Samain, Bievres (FR)

(21) Appl. No.: 13/517,006

(22) PCT Filed: Dec. 16, 2010
(86) PCT No.: PCT/EP2010/069988

§ 371 Date: Oct. 23, 2012

(30) Foreign Application Priority Data
Dec. 17, 2009 (FR) 0959126

(51) Int. Cl. A61K 8/87; A61K 8/90; A61Q 15/00; A61K 8/81

(52) U.S. Cl. 424/65

(57) ABSTRACT

The present invention relates to the use of a cosmetic composition comprising, in a cosmetically acceptable medium, one or more nonionic, anionic, amphoteric or cationic film-forming elastomeric polymers, capable of giving, on drying of the said elastomeric polymer(s), at room temperature and at a relative humidity of 55%, a material with a mechanical profile defined by at least: a) a degree of elongation at break (ε) greater than or equal to 150%, b) an instantaneous recovery (R1) of greater than or equal to 75% after an elongation of 150%, c) a recovery at 300 seconds (R₃₀₀ₛ) of greater than 80%, after an elongation of 150%.

The present invention relates to the use of a cosmetic composition comprising, in a cosmetically acceptable medium, one or more particular film-forming elastomeric polymers for the cosmetic treatment of human perspiration. The invention also relates to a cosmetic process for treating human perspiration using a composition comprising one or more particular film-forming elastomeric polymers.

The armpits and also certain other parts of the body are generally the site of much discomfort that may arise directly or indirectly from perspiration. This perspiration often leads to unpleasant and disagreeable sensations that are mainly due to the presence of sweat resulting from perspiration, which may, in certain cases, make the skin and clothing wet, especially in the region of the armpits or on the back, thus leaving unaesthetic visible marks. Moreover, the presence of sweat generally gives rise to the production of body odours, which are generally unpleasant. Finally, during its evaporation, sweat may also leave salts and/or proteins at the surface of the skin, which may result in whitish marks on clothing. Such discomfort is noticed, including in the case of moderate perspiration.

In the cosmetic field, it is thus well known to use, in topical application, antiperspirant products containing substances that have the effect of limiting or even preventing the flow of sweat in order to overcome the problems mentioned above. These products are generally available in the form of roll-ons, sticks, aerosols or sprays.

Antiperspirant substances are generally formed from aluminium salts, such as aluminium chloride and aluminium hydroxyhalides, or complexes of aluminium and zirconium. These substances reduce the flow of sweat by forming a plug in the sweat duct.

However, the use of these substances at high concentrations, especially in a content ranging from 15% to 20% by weight, for the purpose of obtaining good antiperspirant efficacy, often has the drawback of leading to formulation difficulties.

Furthermore, it has been found that the antiperspirant efficacy of these substances is limited, which means that they need to be applied regularly to the skin in order to obtain a satisfactory effective antiperspirant effect. However, in the case of certain users, repeated application of these substances has the drawback of leading to skin irritation.

Moreover, another drawback associated with the use of these aluminium salts lies in the fact that the antiperspirant effect imparted by such substances generally has a tendency to disappear, especially in the case of successive washing or in the case of heavy perspiration.

Finally, these antiperspirant substances may leave marks during their application to the skin, which has the consequence of staining clothing.

As a variant, it has also been proposed to develop a method that consists in using botulinum toxin to give a satisfactory antiperspirant effect. However, the need to inject such a toxin at each use greatly limits the exploitation of this method.

To overcome all of the drawbacks mentioned above, it has been proposed to seek other effective active substances, which are well tolerated by the skin and easy to formulate, in order to replace all or some of the aluminium salts and/or aluminium and zirconium complexes.

Specifically, limiting the flow of sweat may be achieved by partially obstructing the sweat ducts by means of forming a plug in the sweat duct, but also by forming at the surface of the skin a film that is resistant to sweat. Thus, many approaches directed towards covering the surface of the skin with a film have been developed in order to limit the flow of sweat.

By way of example, patent applications WO 93/24105 and U.S. Pat. No. 5,508,024 describe antiperspirant compositions essentially comprising, in a cosmetically acceptable medium, at least one water-insoluble non-toxic polymer that is capable of forming an antiperspirant occlusive film on the skin. In particular, the water-insoluble polymers used in such compositions are dissolved or dispersed in a medium based on alcohols, especially aliphatic alcohols containing short alkyl chains, and form a flexible occlusive film on the skin.

Patent application US 2003/0 194 387 describes an antiperspirant composition for topical application comprising, in a cosmetically acceptable medium, at least one active antiperspirant substance, which is chosen especially from aluminium salts, and at least one water-insoluble non-toxic polyester that is capable of forming an occlusive film on the skin. The film thus formed, following application of the polyester to the skin, has the purpose of reinforcing the efficacy of the aluminium salts and of being able to formulate compositions with low contents of aluminium salts.

Similarly, patent application U.S. Pat. No. 6,387,356 describes a composition comprising, in a medium comprising alcoholic solvents, at least one cellulose ester that is soluble in an aqueous-alcoholic medium, capable of forming a thin film on the surface of the skin, and at least one antiperspirant substance that is generally chosen from aluminium salts.

Similarly, patent application DE 2 947 060 describes a composition, in particular a composition conditioned in an aerosol device, comprising at least one acrylic polymer capable of forming a film on the skin and at least one antiperspirant substance, which may especially be aluminium salts.

Moreover, patent application WO 95/27473 describes the use of polymers containing quaternized amine groups, for forming films on the skin.

However, these occlusive film-forming polymers do not make it possible to obtain entirely satisfactory antiperspirant efficacy, and still give rise to formulation problems. In particular, the antiperspirant effects imparted by such compositions still remain too limited over time.

Another approach consisted in using compositions comprising fatty substances capable of forming an occlusive layer on the skin. In particular, international patent application WO 00/13653 describes a composition with the consistency of a gel, comprising (a) at least one volatile or non-volatile organosilicon compound and (b) at least one gelling agent comprising a non-volatile organosilicon compound containing an aliphatic hydrocarbon-based chain.

Similarly, patent application WO 96/27364 describes a composition intended to be applied to the skin, comprising an anhydrous vehicle that comprises cosmetic active agents for the skin and that is formed from 5% to 30% by weight of a wax with a high melting point, from 5% to 30% by weight of monoglyceride, from 5% to 60% by weight of petroleum jelly and from 0.1% to 30% by weight of fatty alcohol.

However, the film formed after applying these compositions to the skin has a tendency to degrade easily, which means that the desired antiperspirant effects are not entirely satisfactory.

In order to overcome these drawbacks, a method consisting in using a polymer film that is capable of absorbing sweat was developed. In particular, patent application US 93/87538 describes the use of a polymer such as a cellulose ester, which forms a film on the skin that absorbs perspiration.

However, the film formed on the skin is not entirely satisfactory since the amount of water to be absorbed is generally greater than the amount of water that is absorbable by such compounds.

Moreover, it has also been proposed in international patent application WO 2001/054 658 to use anhydrous, non-adhesive antiperspirant compositions comprising at least one water-reactive cyanoacrylate monomer, an anhydrous medium, a polymerization inhibitor and an active substance chosen from a deodorant substance, an antiperspirant substance and a fragrancing substance, or a mixture of these substances. The cyanoacrylate monomers used in such compositions polymerize anionically directly on the surface of the skin in the presence of a nucleophilic agent, such as hydroxide ions (OH⁻) contained in water, to form a water-resistant polymer film. In other words, the cyanoacrylate monomers react with the sweat to form in situ, via anionic polymerization, a film on the skin that blocks the sweat ducts.

However, these occlusive film-forming polymers do not afford entirely satisfactory antiperspirant efficacy and still pose formulation problems due to the amounts of solvent required to dissolve the cyanoacrylate monomers.

Thus, there is a real need to develop compositions that do not have the drawbacks mentioned above, i.e. that afford a satisfactory antiperspirant effect, especially in terms of efficacy and of resistance to sweat, and that are suitably tolerated by the skin.

The Applicant has discovered, surprisingly, that the use on the skin of one or more particular film-forming elastomeric polymers in cosmetic compositions can lead to the desired properties, i.e. these film-forming elastomeric polymers can efficiently treat human perspiration while at the same time having a toxicological profile that is suitable for the skin, and being easy to formulate in products for reducing perspiration.

Specifically, the use of a cosmetic composition comprising one or more film-forming elastomeric polymers can give a satisfactory antiperspirant effect that is capable of being maintained in the hours following the application, while at the same time giving the skin a dry sensation and giving the user a sensation of comfort. In other words, the use of such a composition makes it possible to keep the skin dry when the user perspires.

In particular, the film-forming elastomeric polymer(s) used lead to the formation on the skin of a coating that shows good adhesion and good flexibility, which gives it high mobility. More particularly, the film-forming elastomeric polymer(s) according to the invention allow the formation of a coating that is capable of being deformed as a function of the movements of the skin and of substantially resuming its initial shape when the deformations cease.

In other words, the Applicant has observed that certain film-forming elastomeric polymers lead to the formation of a coating with advantageous mechanical properties that allow it to be deformed as a function of the movements of the skin and to substantially regain its initial form when these deformations cease, and consequently to satisfactorily treat human perspiration since the coating obtained is remanent.

Moreover, the coating obtained also shows satisfactory resistance, especially relative to the pressure exerted by water droplets, by the movements of the body or by sweat leaving the skin pores.

Thus, the Applicant has observed that the application to the skin of a cosmetic composition comprising one or more elastomeric film-forming polymers makes it possible to obtain a coating which, on the skin, is flexible, adhesive, resistant, capable of becoming deformed as a function of the movements of the skin and of substantially regaining its initial shape when the deformations cease, thus giving it a satisfactory antiperspirant effect.

One subject of the present invention is thus especially the use of a cosmetic composition comprising, in a cosmetically acceptable medium, one or more nonionic, anionic, amphoteric or cationic film-forming elastomeric polymers, which are capable of giving, on drying of the said polymer(s), at room temperature and at a relative humidity of 55%, a material with a mechanical profile defined by at least:

a) a degree of elongation at break (E) of greater than or equal to 150%,

b) an instantaneous recovery (Rᵢ) of greater than or equal to 75% after an elongation of 150%,

c) a recovery at 300 seconds (R₃₀₀ₛ) of greater than 80%, after an elongation of 150%,

for the cosmetic treatment of human perspiration.

The material obtained by drying the said film-forming elastomeric polymer(s) is thus sufficiently extensible so as not to break following deformations caused by the movements of the skin and to regain a shape substantially identical to its initial shape. Thus, the material obtained allows human perspiration to be treated satisfactorily.

In other words, one subject of the present invention is thus especially the use of a cosmetic composition comprising, in a cosmetically acceptable medium, one or more film-forming elastomeric polymers as defined previously for the cosmetic treatment of human perspiration.

In particular, the use of such a cosmetic composition gives an antiperspirant effect.

More particularly, the film-forming elastomeric polymer(s) as defined previously are used as antiperspirant agents.

For the purposes of the present invention, the term “relative humidity” means the ratio of the partial vapour pressure of water contained in the air to the saturating vapour pressure at the same temperature and pressure. This value makes it possible to measure the ratio between the water vapour content of the air and its maximum capacity for containing water under these conditions. The relative humidity is measured using a hygrometer.

For the purposes of the present invention, the term “film-forming polymer” means a polymer that is capable, by itself or in the presence of a film-forming auxiliary, of forming a continuous film that adheres to a support, especially to the skin.

Another subject of the present invention consists of a cosmetic process for treating human perspiration, and possibly body odours related to human perspiration, especially underarm odours, which consists in applying to the skin a cosmetic composition comprising, in a cosmetically acceptable medium, one or more nonionic, anionic, amphoteric or cationic film-forming elastomeric polymers as described above.

Moreover, the present invention also relates to a cosmetic process for treating human perspiration in two steps, comprising a first step (i) that consists in applying to the skin a cosmetic composition comprising, in a cosmetically acceptable medium, one or more film-forming elastomeric polymers as defined previously, and a second step (ii) that consists in reducing any discomfort associated with the elastomeric coating formed on the skin by the film-forming elastomeric polymer(s).

Other subjects, characteristics, aspects and advantages of the invention will emerge even more clearly on reading the description and the examples that follow.

The material obtained, by drying the said film-forming elastomeric polymer(s), at room temperature and at a relative humidity of 55% and having a mechanical profile as described above, constitutes a coating that covers the surface of the area of skin that has been treated with the composition according to the invention.

The coating thus obtained, under the conditions described previously, preferentially has an elastic modulus σ of greater than 10⁵ Pa and even more preferentially an elastic modulus value σ that is greater than 10⁶ Pa, and even more preferentially an elastic modulus σ that is greater than 5×10⁶ Pa.

Preferably, the material obtained, by drying the said film-forming elastic polymer(s), at room temperature and at a relative humidity of 55% and having a mechanical profile as described above, is a film that covers the surface of the area of skin treated with the composition according to the invention.

In particular, the term “film” means a thin, manipulable solid. The term “thin” means a solid having a thickness of at least 1 μm and even more preferentially of at least 5 μm. Such a film may be square, rectangular or disc-shaped, or of any other shape.

The film thus obtained under these conditions generally has a thickness from 2 μm to 1000 μm, preferably from 4 to 200 μm and better still from 8 to 100 μm. It may have an area from 10 to 800 cm² and preferably from 40 cm² to 200 cm².

In particular, the film obtained on the skin is water-insoluble.

For the purposes of the present invention, the elongation at break (ε) of a material defines its capacity to be stretched before breaking when it is placed under a tensile stress. The degree of elongation of the material is measured as a percentage.

For the purposes of the present invention, the instantaneous recovery (Rᵢ) of a material defines its capacity to regain its initial shape or a shape substantially identical to its initial shape after having been deformed following an elongation during a tensile stress. The recovery of the material is also measured as a percentage.

For the purposes of the present invention, the degree of elongation at break and the recovery are evaluated by means of the tensile tests described below.

To perform the tensile tests, a film intended for producing specimens is made by placing in a Teflon mould the sufficient amount of mixture comprising the film-forming elastomeric polymer(s) to obtain a film 500 μm±±50 μm thick. Drying is continued until the weight of the film no longer changes, which may typically take 12 days.

In particular, for the purposes of the present invention, the term “film intended for producing or making specimens” means a film obtained by drying the said film-forming elastomeric polymer(s), at room temperature (22° C.±2° C.) and at a relative humidity of 55%±5%, from a mixture containing at least 3% of active materials, i.e. 3% by weight of film-forming elastomeric polymer relative to the total weight of the mixture.

When the mixture used to produce the film for the manufacture of specimens contains less than 3% by weight of active materials, a preliminary concentration operation is performed, for example by evaporating off some of the solvent so that the mixture contains at least 3% of elastomeric polymers. This operation makes it possible to avoid excessively long drying.

The film obtained is then chopped into rectangular specimens 80 mm long and 15 mm wide.

The tests are performed on a machine sold under the name Lloyd or sold under the name Zwick under the same temperature and humidity conditions as for the drying, i.e. at room temperature (22° C.±2° C.) and at a relative humidity of 55%±5%.

The specimens are drawn at a rate of 20 mm/minute and the distance between the jaws is 50±1 mm.

To determine the instantaneous recovery (Rᵢ), the process is performed as follows:
the specimen is drawn by 150% (εₘₐₓ), i.e. 1.5 times its initial length (I₀),
the stress is removed by imposing a return speed equal to the tensile speed, i.e. 20 mm/minute, and the elongation of the specimen is measured as a percentage, after returning to zero load (εᵢ).

The percentage instantaneous recovery (Rᵢ) is given by the formula below:

Rᵢ=((εₘₐₓ−εᵢ)/εₘₐₓ)×100

To determine the recovery at 300 seconds, the specimen is maintained at zero stress for a further 300 seconds, after having undergone the preceding operations, and its degree of elongation is measured as a percentage (ε₃₀₀ₛ). In other words, the recovery at 300 seconds corresponds to the residual degree of elongation of the specimen 300 seconds after returning to zero load (εᵢ).

Thus, the recovery at 300 seconds (R₃₀₀ₛ) of a material defines its capacity to regain its shape or a shape substantially identical to its initial shape a further 300 seconds after the return to zero load (εᵢ) and after having been deformed following an elongation during a tensile stress.

The percentage recovery at 300 seconds (R₃₀₀ₛ) is therefore given by the formula below:

R₃₀₀ₛ=((εₘₐₓ=εᵢ)/ε₃₀₀ₛ)×100

Advantageously, the film-forming elastomeric polymer(s) according to the invention, optionally combined with other agents such as plasticizers and/or a film-forming agent, are such that they form, under the conditions of the tests described above, a material with a degree of elongation at break (ε) greater than 150%, preferably at least greater than 250% and even more preferentially ranging from 250% to 1000%, an instantaneous recovery (Rᵢ) ranging from 75% to 100% and a recovery at 300 seconds (R₃₀₀ₛ) ranging from 80% to 100% and preferably from 90% to 100%.

The film-forming elastomeric polymer(s) used in the composition according to the invention are structurally chosen from those that are capable of giving, after drying at room temperature and at a relative humidity of 55%, a material having the mechanical profile described previously.

In other words, the chemical nature of the film-forming elastomeric polymer(s) is chosen such that the film-forming elastomeric polymers are capable of giving, under the conditions described previously, a material that has the mechanical properties described previously.

Advantageously, the elastomeric film-forming polymer(s) are chosen from the group comprising polyurethanes, polyvinyl alcohols, polymers comprising at least one (meth)acrylic unit, and combinations thereof. It may be in the form of a homopolymer or a copolymer. In particular, it is in non-crosslinked form in the composition. It may also be in the form of a copolymer whose monomer distribution along the chain is not random. For example, the copolymer may be a block or pseudo-block polymer.

Preferably, the film-forming elastomeric polymer(s) used in the cosmetic composition are nonionic.

Preferably, the film-forming elastomeric polymer(s) used in the cosmetic composition are chosen from copolymers obtained by copolymerization of hexadiol, neopentyl glycol, adipic acid, dicyclohexylmathane diisocyanate, ethylenediamine and sodium N(2-aminoethyl)-3-aminoethanesulfonate and copolymers obtained by copolymerization of adipic acid, hexanediol, neopentyl glycol, hexamethylene diisocyanate, N-(2-amino ethyl)-3-amino ethanesulfonic acid and ethylenediamine.

More preferentially, the film-forming elastomeric polymer(s) used in the cosmetic composition according to the invention are chosen from polyurethanes. In particular, the polyurethanes used in the cosmetic composition according to the invention are those sold under the name Baycusan C1001 or C1004, and more particularly the product sold under the name Baycusan C1001.

The product sold under the name Baycusan C1001 corresponds to a polymer that is formed in a multi-step reaction. Indeed, a copolymer of hexanediol, neopentyl glycol and adipic acid is reacted with hexamethylene diisocyanate. The resulting polymer is further reacted with N-(2-aminoethyl)-3-aminoethanesulfonic acid and ethylenediamine.

More particularly, the film-forming elastomeric polymer(s) used in the cosmetic composition according to the invention are chosen from polyurethanes that are capable of giving, after drying the said polyurethane(s), at room temperature and at a relative humidity of 55%, a material, especially a film, with a degree of elongation at break (ε) of 450%, an instantaneous recovery (Rᵢ) of 88% and a recovery at 300 seconds (R₃₀₀ₛ) of 94%.

Thus, the cosmetic composition according to the invention may advantageously comprise one or more polyurethanes that are capable of giving, under the conditions described previously, a material having the mechanical profile described above.

The elastomeric film-forming polymers used in the compositions according to the invention may be synthesized according to the method described in patent application FR 2 815 350.

According to another particular form of the invention, the elastomeric film-forming polymer is a polyamide-polyether polymer.

### Polyamide-Polyether Polymers

Preferably also, the polymers of the polyamide-polyether type of the invention will be water-insoluble.

According to one particular mode of the invention, the polymers of the polyamide-polyether type of the invention have a permeability to water vapour of greater than 1000 g/m²/24 hours. This permeability is determined by measuring the amount of water vapour in grams that crosses 1 m² of coating in 24 hours, from a wet zone to a dry zone (ASTM E 96 E). The operating conditions adopted are a relative humidity percentage of 90% and a temperature of 38° C. The measurement is performed on a sample of coating 15 μm thick. The permeability measurement may be measured using permeability-measuring equipment such as the equipment bearing the reference Perme™ W3-060 available from the company Labthink Instruments Co. Limited.

According to another particular mode of the invention, the polymers of the polyamide-polyether type of the invention may be characterized by adhesion properties on the skin higher than the adhesion properties obtained with water-resistant elastomeric polymers such as certain dispersions of polyurethane in aqueous phase available especially under the reference Baycusan C1001 from Bayer.

The polyamide-polyether polymers of the invention will preferably be chosen from copolymers containing polyamide blocks (in particular polyamide-6 or polyamide-12) and polyether blocks (in particular polyethylene glycol or polytetramethylene glycol).

The copolymers containing polyamide blocks and polyether blocks according to the invention result from the copolycondensation of polyamide sequences bearing reactive ends with polyether sequences bearing reactive ends, such as, inter alia:

1) Polyamide sequences bearing diamine chain ends with polyoxyalkylene sequences bearing dicarboxylic chain ends.

2) Polyamide sequences bearing dicarboxylic chain ends with polyoxyalkylene sequences bearing diamine chain ends obtained by cyanoethylation and hydrogenation of α,ω-dihydroxylated aliphatic polyoxyalkylene sequences known as polyetherdiols.

3) Polyamide sequences bearing dicarboxylic chain ends with polyetherdiols, the products obtained being, in this particular case, polyetheresteramides. The copolymers of the invention are advantageously of this type. The polyamide sequences bearing dicarboxylic chain ends are derived, for example, from the condensation of polyamide precursors in the presence of a chain-limiting dicarboxylic acid.

The polyamide sequences bearing diamine chain ends originate, for example from the condensation of polyamide precursors in the presence of a chain-limiting diamine.

The polymers bearing polyamide blocks and polyether blocks of the invention may also comprise randomly distributed units. These polymers may be prepared by the simultaneous reaction of the polyether and of the precursors of the polyamide blocks.

For example, it is possible to react polyetherdiol, polyamide precursors and a chain-limiting diacid. A polymer is obtained essentially having polyether blocks and polyamide blocks of very variable length, but also the various reagents that have reacted randomly, which are distributed randomly (statistically) along the polymer chain.

It is also possible to react polyetherdiamine, polyamide precursors and a chain-limiting diacid. A polymer is obtained essentially having polyether blocks and polyamide blocks of very variable length, but also the various reagents that have reacted randomly, which are distributed randomly (statistically) along the polymer chain.

Three types of polyamide block may advantageously be used.

According to a first type, the polyamide sequences originate from the condensation of a dicarboxylic acid and a diamine.

According to a second type, the polyamide sequences result from the condensation of one or more α,ω-aminocarboxylic acids and/or of one or more lactams containing from 6 to 12 carbon atoms in the presence of a dicarboxylic acid containing from 4 to 12 carbon atoms or of a diamine.

According to a third type, the polyamide sequences result from the condensation of at least one α,ω-aminocarboxylic acid (or a lactam), at least one diamine and at least one dicarboxylic acid.

According to one variant of this third type, the polyamide blocks result from the condensation of at least two α,ω-aminocarboxylic acids or from at least two lactams containing from 6 to 12 carbon atoms or from one lactam and one aminocarboxylic acid not having the same number of carbon atoms, in the optional presence of a chain limiter. Advantageously, the polyamide blocks of the second type are polyamide 12 or polyamide 6 blocks. As examples of polyamide sequences of the third type, mention may be made of the following:

a) 6.6/Pip. 10/12 in which 6.6 denotes hexamethyleneadipamide units (hexamethylenediamine condensed with adipic acid). Pip. 10 denotes units resulting from the condensation of piperazine and of sebacic acid. 12 denotes units resulting from the condensation of lauryllactam. The weight proportions are, respectively, 25 to 35/20 to 30/20 to 30; the total being 80, and advantageously 30 to 35/22 to 27/22 to 27; the total being 80. For example, 32/24/24 proportions give a melting point from 122 to 137° C.

b) 6.6/6.10/11/12 in which 6.6 denotes hexamethylenediamine condensed with adipic acid, 6.10 denotes hexamethylenediamine condensed with sebacic acid, and 11 denotes units resulting from the condensation of aminoundecanoic acid, and 12 denotes units resulting from the condensation of lauryllactam. The weight proportions are, respectively, 10 to 20/15 to 25/10 to 20/15 to 25; the total advantageously being 70:12 to 16/18 to 25/12 to 16/18 to 25; the total being 70. For example, 14/21/14/21 proportions lead to a melting point from 119 to 131° C.

The polyamide blocks are obtained in the presence of a diacid or of a chain-limiting diamine if polyamide blocks bearing acid or amine ends are desired. If the precursors already comprise a diacid or a diamine, it suffices, for example, to use it in excess. By way of example of aliphatic α,ω-aminocarboxylic acid, mention may be made of aminocaproic acid, 7-aminoheptanoic acid, 11-aminoundecanoic acid and 12-aminododecanoic acid. As examples of lactams, mention may be made of caprolactam, oenantholactam and lauryllactam. As examples of aliphatic diamines, mention may be made of hexamethylenediamine, dodecamethylenediamine and trimethylhexamethylenediamine. As an example of a cycloaliphatic diacid, mention may be made of 1,4-cyclohexyldicarboxylic acid. As examples of aliphatic diacids, mention may be made of butanedioic acid, adipic acid, azelaic acid, suberic acid, sebacic acid, dodecanedicarboxylic acid, dimerized fatty acids (these dimerized fatty acids preferably have a dimer content of at least 98%; they are preferably hydrogenated; they are sold under the brand name Pripol by the company Unichema, or under the brand name Empol by the company Henkel) and polyoxyalkylene-α,ω-diacids. As examples of aromatic diacids, mention may be made of terephthalic acid (T) and isophthalic acid (I). The cycloaliphatic diamines may be bis(4-aminocyclohexyl)methane (BACM), bis(3-methyl-4-aminocyclohexyl)methane (BMACM), 2-2-bis(3-methyl-4-aminocyclohexyl)propane (BMACP) and para-amino dicyclohexylmethane (PACM) isomers. The other diamines commonly used may be isophoronediamine (IPDA), 2,6-bis(aminomethyl)norbornane (BAMN) and piperazine.

The polyether blocks may represent 5% to 85% by weight of the copolymer bearing polyamide and polyether blocks.

The polyether blocks are formed from alkylene oxide units. These units may be, for example, ethylene oxide units, propylene oxide units or tetrahydrofuran (which leads to polytetramethylene glycol chains). Use is thus made of PEG blocks, i.e. blocks formed from ethylene oxide units, PPG blocks, i.e. blocks formed from propylene oxide units, polytrimethylene glycol ether units (such copolymers with polytrimethylene ether blocks are described in patent U.S. Pat. No. 6,590,065), and PTMG blocks, i.e. blocks formed from tetramethylene glycol units, also known as polytetrahydrofuran.

Use is advantageously made of PEG blocks or of blocks obtained by oxyethylation of bisphenols, for instance bisphenol A. The latter products are described in patent EP 613 919.

The polyether blocks may also be formed from ethoxylated primary amines.

These blocks are also advantageously used. As examples of ethoxylated primary amines, mention may be made of the products of formula:

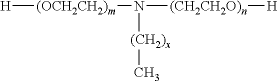

in which m and n are between 1 and 20 and x is between 8 and 18. These products are commercially available under the brand name Noramox® from the company CECA and under the brand name Genamin® from the company Clariant.

The amount of polyether blocks in these copolymers bearing polyamide blocks and polyether blocks is advantageously from 10% to 70% by weight and preferably from 35% to 60% by weight of the copolymer.

The polyetherdiol blocks are either used in unmodified form and copolycondensed with polyamide blocks bearing carboxylic end groups, or they are aminated to be converted into polyetherdiamines and condensed with polyamide blocks bearing carboxylic end groups. They may also be mixed with polyamide precursors and a chain-limiting diacid to make polymers bearing polyamide blocks and polyether blocks having randomly distributed units.

The number-average molar mass Mn of the polyamide sequences is between 500 and 10 000 and preferably between 500 and 4000, except for the polyamide blocks of the second type.

The mass Mn of the polyether blocks is between 100 and 6000 and preferably between 200 and 3000.

These polymers bearing polyamide blocks and polyether blocks, whether they originate from the copolycondensation of polyamide and polyether sequences prepared previously or from a one-step reaction, have, for example, an intrinsic viscosity of between 0.8 and 2.5 measured in meta-cresol at 25° C. for an initial concentration of 0.8 g/100 ml.

As regards the preparation of the copolymers bearing polyamide blocks and polyether blocks, they may be prepared via any means for attaching polyamide blocks and polyether blocks.

In practice, essentially two processes are used, one known as a two-step process, the other a one-step process.

In the two-step process, the polyamide blocks are first made, and in a second step the polyamide blocks and the polyether blocks are then attached.

In the one-step process, the polyamide precursors, the chain limiter and the polyether are mixed together; a polymer essentially having polyether blocks and polyamide blocks of very variable length, but also the various reagents that have reacted randomly, which are distributed randomly (statistically) along the polymer chain is obtained.

Whether it is a one-step or a two-step process, it is advantageous to perform the process in the presence of a catalyst. The catalysts described in patents U.S. Pat. No. 4,331,786, U.S. Pat. No. 4,115,475, U.S. Pat. No. 4,195,015, U.S. Pat. No. 4,839,441, U.S. Pat. No. 4,864,014, U.S. Pat. No. 4,230,838 and U.S. Pat. No. 4,332,920, WO 04 037898, EP 1 262 527, EP 1 270 211, EP 1 136 512, EP 1 046 675, EP 1 057 870, EP 1 155 065, EP 506 495 and EP 504 058 may be used. Polyamide blocks may also be manufactured in the one-step process.

Copolymers bearing polyamide blocks (in particular PA-6 or PA-12) and polyether blocks (in particular polyethylene glycol or polytetramethylene glycol ether) and more particularly those sold under the name Pebax® by the company Arkema, will be more particularly used. Preferably, the polymer is chosen from the references Pebax® 2533 SA 01, Pebax® 2533 SD 02, Pebax® 3533 SA 01 and Pebax® 2533 SP 01. Preferably, the polymer according to the invention is the reference Pebax® 2533 SA 01.

In the compositions in accordance with the invention, the polyamide-polyether polymer(s) are preferably present in a concentration ranging from 0.05% to 20% by weight, more preferentially from 0.1% to 15% by weight and, for example, from 0.25% to 10% by weight relative to the total weight of the composition. The amounts will vary as a function of the desired cosmetic application.

### Organic Solvents

The solvent(s) used in the presence of the polymers of the polyether-polyamide type may be chosen from linear C₁-C₄ monoalcohols, in particular ethanol; C₁-C₃₀ alkanes, for instance isododecanes and paraffins; fluorocarbon hydrocarbons such as 1,1-difluoroethane (152a), 1,1,1,2-tetrafluoroethane (134a), acetone and liquefied gases such as dimethylether.

According to one particular form of the invention, the organic solvent(s) may also be one or more propellants in an aerosol device, which, at the time of use and from the formulation outlet, dissolve the polyamide-polyether polymer as defined previously.

Mention may also be made of fatty alcohols, fatty amides and alkylene carbonates such as propylene carbonate.

In the compositions in accordance with the invention, the organic solvent(s) are preferably present in a concentration ranging from 40% to 99% by weight, more preferentially from 60% to 97% by weight, for example from 80% to 95% by weight, relative to the total weight of the composition. The amounts will vary as a function of the desired cosmetic application.

The coating obtained using the liquid composition comprising at least one polyamide-polyether and at least one organic solvent may be used for treating human perspiration and body odours resulting therefrom, especially in the armpits. The coating thus formed, by virtue of its mechanical properties, withstands the inevitable friction in the region of the armpits.

Another subject of the invention is a process for treating human perspiration and the body odours resulting therefrom, characterized in that a coating formed in situ from a composition comprising, in a cosmetically acceptable medium, at least one polyamide-polyether in solution or in dispersion and at least one organic solvent is applied to the area of the body to be treated.

The composition according to the invention may also contain an agent for treating perspiration and/or a deodorant active agent, which will be described later in detail.

According to another variant, a coating formed in situ from a composition (B) as defined previously, comprising, in a cosmetically acceptable medium, at least one polyamide-polyether in solution or in dispersion and at least one organic solvent, may be applied in a first step to the area of the body to be treated, and, in a second step, a composition (A) comprising, in a cosmetically acceptable medium, at least one agent for treating perspiration, may be applied; the order of application being irrelevant.

Another subject of the invention is thus a cosmetic assembly comprising at least:

(i) a first composition (A) as defined previously;

(ii) a second composition (B) as defined previously.

The elastomeric film-forming polymer or the mixture of elastomeric film-forming polymers is preferably present in a concentration ranging from 0.05% to 20% by weight, more preferentially from 0.1% to 15% by weight, for example from 0.25% to 10% by weight, relative to the total weight of the composition.

If necessary, the composition may also comprise one or more plasticizers and/or one or more agents that facilitate the formation of a film from the film-forming elastomeric polymer(s) on the surface of the skin, the function of which is to modify the properties of the elastomeric polymer(s).

Such a film-forming auxiliary may be chosen from any compound known to those skilled in the art as being capable of satisfying the desired function, and may be chosen especially from plasticizers and coalescers.

The elastomeric film-forming polymer(s), optionally combined with one or more plasticizers and/or one or more agents for facilitating the formation of a film are capable of forming a film, after evaporation of the cosmetically acceptable medium. This evaporation may be performed in the open air or by supplying heat, for example using a hairdryer.

As examples of plasticizers and/or agents for facilitating the formation of a film on the surface of the skin, those described in document FR-A-2 782 917 may be used.

In particular, the plasticizer(s) and/or the agent(s) for facilitating the formation of a film on the surface of the skin may be chosen from the usual plasticizers or coalescers, such as:
glycols and derivatives thereof such as diethylene glycol ethyl ether, diethylene glycol methyl ether, diethylene glycol butyl ether or diethylene glycol hexyl ether, ethylene glycol ethyl ether, ethylene glycol butyl ether, ethylene glycol hexyl ether or pentylene glycol,
glycerol esters,
propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,
acid esters, especially carboxylic acid esters, such as citrates, phthalates, adipates, carbonates, tartrates, phosphates, sebacates, or mineral coalescers such as zinc, aluminium, calcium, magnesium or manganese salts,
mixtures thereof.

The plasticizer(s) may also give the coating obtained a water-impermeable nature.

The amount of plasticizer and/or of film-forming agent may be chosen by a person skilled in the art on the basis of his general knowledge, so as to obtain a polymeric system (elastomeric polymers+plasticizer and/or film-forming agent) leading to a film having the desired mechanical properties, while at the same time conserving the composition's desired cosmetic properties. In practice, this amount ranges from 0.01% to 25% of the total weight of the composition and better still from 0.01% to 15% relative to the total weight of the composition.

Advantageously, in the compositions according to the invention, the film-forming polymer is preferably soluble in an organic medium such as an alcoholic medium or is water-dispersible in water or in an aqueous-alcoholic medium.

Preferably, the film-forming polymer is soluble in an organic solvent.

The cosmetically acceptable medium may thus be formed by water and/or one or more solvents such as monoalcohols, polyols or ethers of these alcohols or polyols. Examples that may be mentioned include ethanol, isopropanol, glycerol, propylene glycol or propylene glycol monomethyl ether.

For the purposes of the present invention, the term “compounds that are soluble” in a given medium means polymers which, when introduced into the said medium at 25° C., at a weight concentration equal to 10%, neutralized, if need be, make it possible to obtain a macroscopically homogeneous and transparent solution, i.e. a solution with a light transmittance value, at a wavelength equal to 500 nm, through a sample 1 cm thick, of at least 70% and preferably of at least 80%.

The term “compounds that are dispersible” in a medium means polymers which, when introduced into the said medium at 25° C. at a weight concentration greater than or equal to 1% by weight, make it possible to obtain a non-transparent or translucent homogeneous dispersion when the particles in dispersion are of small size, in particular less than 50 nanometres.

Preferably, the polymer or at least one of the polymers in dispersion or dissolved have a glass transition temperature (Tg) of less than 80° C. and preferably less than 40° C.

Preferably, the cosmetic composition according to the invention is either an aqueous-alcoholic medium, in particular the medium contains water and ethanol, more particularly 20% by weight of water and 50% by weight of ethanol relative to the total weight of the composition, or an alcoholic medium, in particular comprising less than 5% by weight of water.

According to one embodiment, the material obtained, after drying the film-forming elastomeric polymer(s) according to the invention, under the conditions described previously, may be water-impermeable.

In this case, the film-forming elastomeric polymer(s) are preferably not water-soluble.

As a variant, the cosmetic composition according to the invention may also comprise one or more water-insoluble compounds such as oils, waxes or silicones, so as to make the coating obtained water-impermeable.

According to another embodiment, the material obtained, after drying the film-forming elastomeric polymer(s) according to the invention, under the conditions described previously, may be adhesive.

In this case, the cosmetic composition according to the invention may also comprise one or more pressure-sensitive adhesives.

The film-forming elastomeric polymer(s) used in the composition according to the invention may react with the skin by means of a covalent or physical reaction.

The film-forming elastomeric polymer(s) used in the cosmetic composition according to the invention may form a coating of elastomeric nature 30 minutes after evaporation of the cosmetically acceptable medium. As indicated previously, this evaporation of the cosmetically acceptable medium may be performed in the open air or by performing a physical treatment, for example by means of heat or light irradiation.

The cosmetic composition according to the invention may also contain one or more other polymers for regulating the mechanical strength, the instantaneous recovery and/or the recovery at 300 seconds.

In particular, the cosmetic composition according to the invention may comprise one or more film-forming polymers not having any elastomeric nature.

More particularly, the cosmetic composition according to the invention may comprise one or more film-forming polymers, which may be anionic, chosen from copolymers of acrylic acid such as copolymers derived from crotonic acid such as vinyl acetate/vinyl tert-butylbenzoate/crotonic acid terpolymers and crotonic acid/vinyl acetate/vinyl neododecanoate terpolymers sold under the name Resin 28-29-30 by the company National Starch.

In this case, the coating obtained, after drying the said polymer(s), i.e. the film-forming elastomeric polymers and the film-forming non-elastomeric polymers, under the conditions described previously, may have an elongation at break (ε) of at least 250%, an instantaneous recovery (Rᵢ) and a recovery at 300 seconds of at least 80%.

The cosmetic composition according to the invention may also contain particles that may be in the form of platelets or fibres.

The cosmetic composition according to the invention may also comprise agents for improving the adhesion of the coating to the skin, opacifiers, stabilizers, moisturizers, bactericides, preserving agents, nacreous agents, pigments, dyes, fragrances, thickeners, calmatives, refreshing agents, antiperspirant agents such as aluminium salts, odour absorbers such as cyclodextrin, agents for absorbing water and moisture such as calcium salts, agents for absorbing fatty substances, in particular oils, antibacterial agents, preserving agents, antiseptics, antibiotics, destructuring solvents such as urea, moisturizers, photoprotective agents, hair removers, bleaching agents, self-tanning agents, depigmenting agents or any other ingredient usually used in cosmetics for this type of application.

Needless to say, a person skilled in the art will take care to select this or these optional additional compounds such that the advantageous properties intrinsically associated with the cosmetic composition in accordance with the invention are not, or are not substantially, adversely affected by the envisaged addition(s).

The cosmetic composition according to the invention may especially be in the form of a gel, a cream, a liquid, a dispersion, a suspension, an emulsion, especially an oil-in-water (O/W) or water-in-oil (W/O) emulsion, or a multiple emulsion (W/O/W or polyol/O/W or O/W/O), a stick or a spray.

In particular, the composition according to the invention may be pressurized and may be conditioned in an aerosol device formed by: (A) a container comprising an antiperspirant composition comprising one or more film-forming elastomeric polymers as defined above, (B) at least one propellant and a means for dispensing the said aerosol composition.

The propellants generally used in products of this type, and which are well known to those skilled in the art, are, for instance, dimethyl ether (DME); volatile hydrocarbons such as n-butane, propane, isobutane, and mixtures thereof, optionally with at least one chloro and/or fluoro hydrocarbon; among the latter, mention may be made of the compounds sold by the company DuPont de Nemours under the names Freon® and Dymel®, and in particular mono fluorotrichloromethane, difluoro dichloromethane, tetrafluorodichloroethane and 1,1-difluoroethane sold especially under the trade name Dymel 152 A by the company DuPont. Carbon dioxide, nitrous oxide, nitrogen or compressed air may also be used as propellant.

The dispensing means, which forms part of the aerosol device, is generally formed by a dispensing valve controlled by a dispensing head, itself comprising a nozzle via which the aerosol composition is vaporized. The container containing the pressurized composition may be opaque or transparent. It may be made of glass, of polymer or of metal, optionally covered with a protective varnish coat.

Thus, the composition according to the present invention may be conditioned in pressurized form in an aerosol device or in a pump-dispenser bottle, conditioned in a device equipped with a ball applicator (roll-on).

Such a composition contains in this regard the ingredients generally used in products of this type, which are well known to those skilled in the art.

The composition may thus be conditioned in applicators of roll-on type or sprays, or alternatively applied onto wipes.

According to one particular embodiment, the composition according to the invention may be in the form of a coating that is formed from the film-forming elastomeric polymer(s) so as to be able to be transferred by friction or by contact with the skin.

In this case, the coating is preferably arranged on a support that may be a flexible or inflexible article, for instance a sheet or a fabric. The surface of the support is made of a sparingly adhesive material or treated so that it is sparingly adhesive.

The surface of the coating may also be treated with an adhesive.

The composition according to the invention may be provided in leaktight conditioning, in particular with some of the solvent or fluid capable of penetrating the skin.

Thus, once removed from the conditioning, the composition is applied to the skin in combination with the solvent or the fluid. After evaporating off the solvent or the fluid, this results in the formation of an elastomeric coating on the skin.

The present invention also relates to a cosmetic process for treating human perspiration, and optionally the body odours associated with human perspiration, especially underarm odours, which consists in applying to the skin a cosmetic composition comprising, in a cosmetically acceptable medium, one or more film-forming elastomeric polymers as described previously.

In particular, the cosmetic composition comprising one or more film-forming elastomeric polymers may be applied to the skin several times.

Preferably, the cosmetic composition according to the invention is not rinsed off after application.

According to one embodiment, the treatment process according to the present invention also relates to a cosmetic process for treating human perspiration in two steps, comprising (i) a step consisting in applying to the skin a cosmetic composition A comprising one or more film-forming elastomeric polymers as described previously, and (ii) a step consisting in mechanically reinforcing the material obtained by the film-forming elastomeric polymer(s) according to the invention.

The second step (ii) may consist in applying a cosmetic composition B comprising one or more film-forming polymers, which may be anionic, chosen from copolymers derived from crotonic acid such as vinyl acetate/vinyl tert-bytylbenzoate/crotonic acid terpolymers and crotonic acid/vinyl acetate/vinyl neododecanoate terpolymers sold under the name Resin 28-29-30 by the company National Starch, polymers derived from maleic, fumaric or itaconic acid or anhydride with vinyl esters, vinyl ethers, vinyl halides, phenylvinyl derivatives, acrylic acid and esters thereof such as the methyl vinyl ether/monoesterified maleic anhydride copolymer sold under the name Gantrez ES 425 by the company ISP.

Thus, the use of the cosmetic composition B makes it possible to mechanically reinforce the material obtained with the film-forming elastomeric polymer(s) according to the invention.

According to another embodiment, the treatment process according to the present invention also relates to a cosmetic process for treating human perspiration in two steps, comprising (i) a step consisting in applying to the skin a cosmetic composition A comprising one or more film-forming elastomeric polymers as described previously, and (ii) a step consisting in making the material obtained with the film-forming elastomeric polymer(s) according to the invention adhesive.

Thus, the second step (ii) of this process may consist in giving or improving the adhesive nature of the material obtained after the formation on the skin of the material obtained with the film-forming elastomeric polymer(s) according to the invention.

The second step (ii) may consist in applying a cosmetic composition B comprising one or more pressure-sensitive adhesives.

For the purposes of the present invention, the pressure-sensitive adhesives according to the invention are compounds that give the support coated therewith immediate tacky power at room temperature, which allows its instantaneous adhesion to a substrate under the effect of a gentle and brief pressure. Even more particularly, the pressure-sensitive adhesives according to the invention are compounds that have immediate tacky power at room temperature and that adhere to a surface by simple contact without the need for more than the pressure of a finger or a hand.

The pressure-sensitive adhesives used in the present invention are compounds that comprise one or more adhesive organic polymers. In other words, such compounds are made from one or more adhesive organic polymers.

In particular, the pressure-sensitive adhesives used in the present invention may be chosen from adhesive organic polymers and particles comprising at least a first solid phase covered on at least part of its surface with one or more adhesive organic polymers.

The adhesive nature of an organic polymer is generally associated with its glass transition temperature. A necessary but insufficient condition for a polymer to be adhesive is that it must have a glass transition temperature (Tg) that is significantly below room temperature, i.e. below a temperature equal to 25° C.

The adhesive organic polymers used in the present invention preferably have a glass transition temperature (Tg) of less than or equal to 10° C. and preferably less than or equal to 0° C.

The glass transition temperature (Tg) of the adhesive organic polymers according to the present invention may be measured by differential calorimetric analysis (Differential Scanning calorimetry, DSC) under the following conditions:

To measure the glass transition temperature, a film about 150 mm thick of test polymer is prepared by depositing an aqueous solution or dispersion of the polymer in a circular Teflon die 40 mm in diameter and leaving the deposit to dry. The film is dried in an oven at a temperature of about 23° C. under a relative humidity of 45%, until the weight no longer changes. About 5 to 15 mg of the film are taken up and placed in a crucible, which is then introduced into the analyser. The thermal analyser is a DSC-2920 model from the company TA Instruments. The initial and final temperatures of the temperature sweep are chosen so as to surround the desired glass transition temperature. The temperature sweep is performed at a rate of 10° C./minute.

This analysis is performed according to ASTM standard D 3418-97 apart from the above changes.

In particular, the adhesive organic polymers according to the invention may be chosen from adhesive polyesters containing one or more sulfonic functions, cationic or amphoteric polyurethanes comprising one or more self-adhesive tertiary or quaternary amine functions and self-adhesive cationic or amphoteric radical polymers.

Preferably, the adhesive organic polymers according to the invention may be chosen from adhesive polyesters containing one or more sulfonic functions, in particular from adhesive branched polyesters containing one or more sulfonic functions.

More preferentially, the adhesive organic polymer according to the present invention corresponds to the branched sulfonic polyester sold by the company Eastman AQ1350 under the name AQ 1350.

According to another embodiment, the present invention also relates to a cosmetic process for treating human perspiration in two steps, comprising (i) a step consisting in applying to the skin a cosmetic composition A comprising one or more film-forming elastomeric polymers as defined previously for giving an antiperspirant effect, and (ii) a step consisting in reducing the discomfort associated with the elastomeric coating formed on the skin by the film-forming elastomeric polymer(s).

In other words, the second step (ii) makes it possible to minimize any discomfort associated with the film-forming elastomeric polymers once these compounds form an elastomeric coating that adheres to the skin.

In accordance with this embodiment, the second step (ii) of the process may consist in chemically or physically treating the skin.

In particular, the second step (ii) of the process may consist in applying to the skin a cosmetic composition B capable of reducing the discomfort caused by depositing the film-forming elastomeric polymer(s) or in physically treating the skin to reduce such discomfort.

More particularly, the second step (ii) of the process consists in applying to the skin a cosmetic composition B capable of reducing the discomfort caused by depositing the film-forming elastomeric polymer(s).

Thus, the first step (i) may consist in applying to the skin a cosmetic composition A comprising one or more film-forming elastomeric polymers to give an antiperspirant effect, and the second step (ii) may consist in applying a cosmetic composition B that may be a powder, a gel or an oil.

The first step (i) may also consist in applying to the skin a cosmetic composition A comprising one or more film-forming elastomeric polymers and one or more expandable particles capable of swelling in the presence of water, and the second step (ii) may consist in applying water to the skin.

As indicated above, the second step (ii) of the process may consist in physically treating the skin.

The physical treatment of the skin may be performed by raising the temperature of the skin, after applying the cosmetic composition A comprising one or more film-forming elastomeric polymers, by means of an external source of heat. In this case, the second step (ii) of the process consists in circulating heat on contact with the skin by means of an external source of heat that may be a device capable of generating heat. In other words, the second step (ii) of the process then consists of a heat treatment.

In the context of this heat treatment, devices for raising the temperature of the skin from 5° C. to 50° C. are preferably used.

The physical treatment of the skin may also be performed by irradiating the skin by means of a light source after applying the cosmetic composition A comprising one or more film-forming elastomeric polymers. The second step (ii) of the process then consists of a light treatment.

In the context of this light treatment, energies ranging from 0.001 J to 20 J and preferably ranging from 0.04 J to 4 J are preferably used. The exposure of the skin to this light treatment may range from a few seconds to several tens of minutes.

Thus, the first step (i) may consist in applying to the skin a cosmetic composition A comprising one or more film-forming elastomeric polymers, one or more waxes and/or one or more crystalline polymers, and the second step (ii) may consist in raising the temperature of the skin by means of a source of heat after applying composition A so as to melt the wax(es) and/or the crystalline polymer(s) at the surface in order to form a layer on the elastomeric coating formed by the film-forming elastomeric polymer(s) according to the invention and/or inclusions in the elastomeric coating.

In particular, the wax(es) that may be used in composition A described above may be of natural origin or of synthetic origin and may have melting points that may be less than 80° C. Thus, for example, the wax(es) may be fatty alcohols containing from 8 to 40 carbon atoms, ethoxylated C₁₂-C₂₂ fatty alcohols comprising from 2 to about 30 mol of ethylene oxide, fatty acid esters and amides of monohydroxy and polyhydroxy fatty acids containing from 10 to 40 carbon atoms, and triglycerides, all these compounds being concrete at 25° C., silicone wax, beeswax, paraffin wax and isoparaffin wax. Examples of such systems are especially described in patent applications WO 01/13871, pages 9-10, WO 01/07007, page 29, or WO 00/74643, pages 26-29, the content of which forms an integral part of the present invention.

Similarly, the first step (i) may consist in applying to the skin a cosmetic composition A comprising one or more film-forming elastomeric polymers and one or more photosensitive materials, and the second step (ii) may consist in irradiating the skin so as to crosslink the photosensitive material(s).

As a variant, the first step (i) may consist in applying to the skin a cosmetic composition A comprising one or more film-forming elastomeric polymers and one or more expandable particles capable of swelling under the action of heat, and the second step (ii) may consist in raising the temperature of the skin by means of a source of heat so as to make the expandable particle(s) swell.

The expandable particles that are capable of swelling under the action of heat may be in the form of heat-expandable particles.

The term “heat-expandable” more particularly denotes particles that are capable of becoming deformed and of expanding under the action of heat.

The expandable particles that are capable of swelling under the action of heat may be expanded powders such as hollow microspheres, and especially microspheres formed from a terpolymer of vinylidene chloride, acrylonitrile and methacrylate, sold under the name Expancel by the company Kemanord Plast under the references 551 DE 12 (particle size of about 12 μm and density of 40 kg/m³), 551 DE 20 (particle size of about 30 μm and density of 65 kg/m³), 551 DE 50 (particle size of about 40 μm), or the micropearls sold under the name Micropearl F 80 ED by the company Matsumoto.

According to one variant, the present invention relates to a cosmetic process for treating human perspiration in two steps, comprising (i) a step consisting in applying to the skin a cosmetic composition A comprising one or more film-forming elastomeric polymers to give an antiperspirant effect and (ii) a step consisting in reducing the discomfort associated with the application of the film-forming elastomeric polymers and in reinforcing the antiperspirant effect.

In this case, the second step (ii) of the process may consist in applying to the skin a cosmetic composition B that is capable not only of reducing the discomfort caused by the deposition of the film-forming elastomeric polymer(s), but also of reinforcing the antiperspirant effect.

Thus, the first step (i) may consist in applying to the skin a cosmetic composition A comprising one or more film-forming elastomeric polymers, and the second step (ii) may consist in applying to the skin a cosmetic composition B comprising one or more antiperspirant active agents.

In particular, the antiperspirant active agent(s) that may be used in the cosmetic composition B are chosen from aluminium salts, in particular aluminium chlorohydrates or complexes thereof.

Among the aluminium chlorohydrates and complexes containing them used in the antiperspirant sticks of the present invention, mention may be made of the substances used and approved by the F.D.A. (Food & Drug Administration) below: aluminium chlorohydrate, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, aluminium zirconium octachlorohydrate, aluminium zirconium octachlorohydrex GLY, aluminium zirconium pentachlorohydrate, aluminium zirconium pentachlorohydrex GLY, aluminium zirconium tetrachlorohydrate, aluminium zirconium trichlorohydrate, aluminium zirconium tetrachlorohydrex GLY, and aluminium zirconium trichlorohydrex-GLY, given that the abbreviations PEG, PG and GLY denote, respectively, polyethylene glycol, propylene glycol and glycine. Commercial products of this type are sold, for example, by the company Clariant under the name Locron S (aluminium chlorohydrate), by the company Reheis under the name Reach 301 or by the company Guilini Chemie under the name Aloxicoll PF 40 (aluminium chlorohydrex), by the company Reheis under the name Rezal 67 Solution (aluminium zirconium pentachlorohydrate sold as an aqueous solution containing 40% active material).

The said aluminium chlorohydrates and complexes thereof are present in the sticks according to the present invention in concentrations ranging from about 1% to 50% by weight and preferably from about 1% to 30% by weight of active materials (on the basis of the anhydrous aluminium salt excluding the water and the complexing agents) relative to the total weight of composition B.

Similarly, the first step (i) may consist in applying to the skin a cosmetic composition A comprising one or more film-forming elastomeric polymers, and the second step (ii) may consist in applying to the skin a cosmetic composition B comprising one or more absorbing agents, in particular agents capable of absorbing odours or moisture.

The first step (i) may also consist in applying to the skin a cosmetic composition A comprising one or more film-forming elastomeric polymers, and the second step (ii) may consist in applying to the skin a cosmetic composition B comprising one or more fragrancing substances.

The first step (i) may also consist in applying to the skin a cosmetic composition A comprising one or more film-forming elastomeric polymers and one or more photosensitive or heat-sensitive materials and the second step (ii) may consist in raising the temperature of the skin by means of a source of heat or irradiating the skin so as to melt or crosslink the photosensitive material in order to produce a layer that becomes superposed on the adhesive coating formed by the pressure-sensitive adhesive(s) according to the invention.

Specifically, the photosensitive materials will melt or become crosslinked so as to form a layer that becomes superposed on the elastomeric coating formed by the pressure-sensitive adhesive(s), which increases the occlusive effect of the said elastomeric coating.

The first step (i) may also consist in applying to the skin a cosmetic composition A comprising one or more film-forming elastomeric polymers, one or more antiperspirant active agents and/or one or more absorbing agents and/or one or more fragrancing substances, the said antiperspirant active agents, absorbents and/or fragrancing substances being introduced into a hollow material capable of swelling under the action of heat, and the second step (ii) may consist in irradiating the skin or in raising the temperature of the skin after applying composition A.

According to another variant, the present invention relates to a cosmetic process for treating human perspiration in two steps, comprising (i) a step that consists in applying to the skin a cosmetic composition A comprising one or more film-forming elastomeric polymers to give an antiperspirant effect, and (ii) a step also consisting in optionally improving the appearance of the skin.

In this case, the first step (i) may consist in applying to the skin a cosmetic composition A comprising one or more film-forming elastomeric polymers, and the second step (ii) may consist in applying a composition B comprising one or more dyes and/or pigments.

The dye(s) and/or pigment(s) used in composition B are used to obtain a coloration close to that of the skin.

As a variant, the first step (i) may consist in applying to the skin a cosmetic composition A comprising one or more film-forming elastomeric polymers, and the second step (ii) may consist in applying a film with an appearance similar to that of the skin or an anti-reflection film.

The cosmetic compositions B used during the second step (ii) may be conditioned in pressurized form in an aerosol device, in a pump-dispenser bottle, conditioned in a device equipped with a ball applicator (roll-on), or may be in the form of fabrics or films.

The cosmetic compositions B used during the second step (ii) may be applied directly onto the skin or through the clothing.

The cosmetic compositions A and B may be applied several times to the skin. Preferably, the cosmetic compositions A and B are applied to the skin separately or staggered over time.

When the cosmetic compositions A and B are applied to the skin separately, the interval between the application of cosmetic composition A and the application of cosmetic composition B may be between 1 second and 1 hour, preferably between 10 seconds and 20 minutes and even more preferentially between 1 minute and 6 minutes.

According to one embodiment, the skin may be rinsed between the application of the cosmetic composition A and the application of cosmetic composition B.

Preferably, the skin is not rinsed between the application of cosmetic composition A and the application of cosmetic composition B.

It should be noted that when the cosmetic compositions A and B are applied separately, the said cosmetic compositions may be in identical galenical forms or different galenical forms.

When the cosmetic compositions A and B are applied to the skin staggered over time, cosmetic composition A may be applied in the morning and cosmetic composition B may be applied in the evening.

The examples that follow serve to illustrate the present invention.

### EXAMPLES

### Example 1

a) Composition (A) is prepared from the ingredients indicated in the table below, the amounts of which are expressed on a weight basis, unless otherwise indicated.

| Composition A | |
|---|---|
| Polyurethane sold under the name Baycusan C1001 | 30% AM |
| by the company Bayer | |
| Ethanol | 20% |
| Water | 50% |

Composition A leads to the formation of a film, after drying, at room temperature and at a relative humidity of 55%, with a degree of elongation at break (ε) of 450%, an instantaneous recovery (Rᵢ) of 88% and a recovery at 300 seconds (R₃₀₀ₛ) of 94%.

4 g of composition (A) are applied to the left armpit, and are then left to dry.

At the end of the day, the perspiration generated on the left armpit, which was treated, and the perspiration generated on the right armpit, which was not treated, is compared.

It is found at the end of the day that a satisfactory antiperspirant effect is obtained on the left armpit relative to the untreated right armpit. Specifically, the left armpit, which was treated with the composition according to the invention, is less moist than the untreated right armpit.

b) On a model, 1 g of composition (A) is applied to the left arm over an area of about 10 cm². On the other arm, 1 g of a composition comprising aluminium salts was applied over an identical area, i.e. 10 cm².

After a few minutes, the model enters a sauna (hot chamber heated to a temperature 48° C.) for 10 minutes.

The perspiration is then evaluated by placing on each treated region a sheet of absorbent paper that darkens when it is impregnated with sweat.

The evaluation shows that the absorbent paper is very sparingly impregnated in the region of the left arm that was treated with the composition comprising the film-forming elastomeric polymer, relative to the region of the right arm that was treated with the composition comprising the aluminium salts.

Moreover, the region of the left arm that was treated with the composition comprising the film-forming elastomeric polymer is markedly dryer than the region of the right arm treated with the composition comprising the aluminium salts.

### Example 2

a) Composition (B) is prepared from the ingredients indicated in the table below, the amounts of which are expressed by weight, unless otherwise indicated.

| Composition B | |
|---|---|
| Polyurethane sold under the name Baycusan C1001 | 30% AM |
| by the company Bayer | |
| Polymer sold under the name Luviset Si Pur by the | 4% |
| company BASF | |
| Tributyl acetyl citrate | 3% |
| Ethanol | 20% |
| Water | 50% |

Composition B leads to the formation of a film, after drying, at room temperature and at a relative humidity of 55%, with a degree of elongation at break (ε) of 250%, an instantaneous recovery (Rᵢ) and a recovery at 300 seconds (R₃₀₀ₛ) of greater than 80%.

4 g of composition (B) are applied to the left armpit and are then left to dry.

At the end of the day, the perspiration generated on the left armpit, which was treated, and the perspiration generated on the right armpit, which was not treated, are compared.

It is found, at the end of the day, that a satisfactory antiperspirant effect is obtained on the left armpit relative to the untreated right armpit. Specifically, the left armpit, which was treated, does not smell, whereas the untreated right armpit gives off an unpleasant odour.

b) On a model, 1 g of composition (B) is applied to the left arm over an area of about 10 cm². On the other arm, 1 g of a composition comprising aluminium salts was applied over an identical area, i.e. 10 cm².

After a few minutes, the model enters a sauna (hot chamber heated at a temperature of 48° C.) for 10 minutes.

The perspiration is then evaluated by placing on each treated region a sheet of absorbent paper that darkens when it is impregnated with sweat.

The evaluation shows that the absorbent paper is very sparingly impregnated in the region of the left arm that was treated with the composition comprising the film-forming elastomeric polymer, relative to the region of the right arm that was treated with the composition comprising the aluminium salts.

Moreover, the region of the left arm that was treated with the composition comprising the film-forming elastomeric polymer is markedly dryer than the region of the right arm treated with the composition comprising the aluminium salts.

### Example 3

Composition (C) is prepared from the ingredients indicated in the table below, the amounts of which are expressed by weight, unless otherwise indicated.

Composition C

| Polyurethane sold under the name Baycusan C1001 by | 25% AM |
|---|---|
| the company Bayer | |
| Adhesive polymer sold under the name AQ 1350 by the | 5% |
| company Eastman Chemicals | |
| Ethanol | 20% |
| Water | 50% |

Composition C leads to the formation of a film, after drying, at room temperature and at a relative humidity of 55%, with a degree of elongation at break (ε) of 500%, an instantaneous recovery (Rᵢ) of greater than 85% and a recovery at 300 seconds (R₃₀₀ₛ) of greater than 90%.

On a model, 1 g of composition (C) is applied to the left arm over an area of about 10 cm². On the other arm, 1 g of a composition comprising aluminium salts was applied over an identical area, i.e. 10 cm².

After a few minutes, the model enters a sauna (hot chamber heated at a temperature of 48° C.) for 10 minutes.

The perspiration is then evaluated by placing on each treated region a sheet of absorbent paper that darkens when it is impregnated with sweat.

The evaluation shows that the absorbent paper is very sparingly impregnated in the region of the left arm that was treated with the composition comprising the film-forming elastomeric polymer, relative to the region of the right arm that was treated with the composition comprising the aluminium salts.

Moreover, the region of the left arm that was treated with the composition comprising the film-forming elastomeric polymer is markedly dryer than the region of the right arm treated with the composition comprising the aluminium salts.

### Example 4

Composition (D) is prepared from the ingredients indicated in the table below, the amounts of which are expressed on a weight basis, unless otherwise indicated.

| Composition D | |
|---|---|
| Polyurethane sold under the name Baycusan C1004 | 25% AM |
| by the company Bayer | |
| Ethanol | 20% |
| Water | 50% |

Composition D is conditioned in an aerosol device sold under the name EP Spray. This device comprises an assembly formed from a pocket hermetically welded to a valve and a swirling-nozzle diffuser. The valve is attached to a standard aerosol can.

The pocket is filled with composition D and compressed air is introduced between the pocket and the can at a sufficient pressure to make the product exit in spray form. The pressure of the compressed gas is 10 bar.

The composition is sprayed onto the armpits and onto the fabric of the clothing facing the armpit. A satisfactory antiperspirant effect is obtained.

### Example 5

a) Composition (A) is prepared from the ingredients indicated in the table below, the amounts of which are expressed on a weight basis, unless otherwise indicated.

| Composition A | |
|---|---|
| Polyurethane sold under the name Baycusan C1001 | 30% AM |
| by the company Bayer | |
| Ethanol | 20% |
| Water | 50% |

Composition (E) is prepared from the ingredients indicated in the table below, the amounts of which are expressed on a weight basis, unless otherwise indicated.

| Composition E | |
|---|---|
| Adhesive polymer sold under the name AQ 1350 by | 10% AM |
| the company Eastman Chemicals | |
| Ethanol | 20% |
| Water | 70% |

On a model, 2 g of composition (A) are applied to the left armpit and are then left to dry. After 5 minutes, 2 g of composition (E) are applied.

On the same model, 4 g of a composition comprising aluminium salts are applied to the right armpit.

After a few minutes, the model enters a sauna (hot chamber heated at a temperature of 48° C.) for 10 minutes.

The perspiration is then evaluated by placing on each treated region a sheet of absorbent paper that darkens when it is impregnated with sweat.

The evaluation shows that the absorbent paper is very sparingly impregnated at the left armpit that was treated with compositions (A) and then (E), relative to the right armpit treated with the composition comprising aluminium salts.

It is thus observed that a satisfactory antiperspirant effect is obtained.

b) Similarly, 2 g of composition (E) described in Example 5a) are applied to another model on the left armpit and are then left to dry. After 5 minutes, 2 g of composition (A) are applied.

On the same model, 4 g of a composition comprising aluminium salts are applied to the right armpit.

After a few minutes, the model enters a sauna (hot chamber heated to at a temperature of 48° C.) for 10 minutes.

The perspiration is then evaluated by placing on each treated region a sheet of absorbent paper that darkens when it is impregnated with sweat.

The evaluation shows that the absorbent paper is very sparingly impregnated on the left armpit that was treated with compositions (E) and then (A), relative to the right armpit treated with the composition comprising aluminium salts.

### Example 6

a) Composition (A) is prepared from the ingredients indicated in the table below, the amounts of which are expressed on a weight basis, unless otherwise indicated.

| Composition A | |
|---|---|
| Polyurethane sold under the name Baycusan C1001 | 30% AM |
| by the company Bayer | |
| Ethanol | 20% |
| Water | 50% |

Composition (F) is prepared from the ingredients indicated in the table below, the amounts of which are expressed on a weight basis, unless otherwise indicated.

| Composition F | |
|---|---|
| Film-forming anionic polymer sold under the name | 16% AM |
| Luviset Si Pur by the company BASF | |
| Glycerol | 6% |
| Ethanol | 26% |
| Water | 70% |

2 g of composition (A) are applied to the left armpit and are then left to dry. After five minutes, 2 g of composition (F) are applied.

On the same model, 4 g of a composition comprising aluminium salts are applied to the right armpit.

After a few minutes, the model enters a sauna (hot chamber heated at a temperature of 48° C.) for 10 minutes.

The perspiration is then evaluated by placing on each treated region a sheet of absorbent paper that darkens when it is impregnated with sweat.

The evaluation shows that the absorbent paper is very sparingly impregnated on the left armpit that was treated with compositions (A) and then (F), relative to the right armpit treated with the composition comprising aluminium salts.

It is thus observed that a satisfactory antiperspirant effect is obtained.

b) Similarly, on another model, 2 g of composition (F) described in Example 6a) are applied to the left armpit and are then left to dry. After 5 minutes, 2 g of composition (A) are applied.

On the same model, 4 g of a composition comprising aluminium salts are applied to the right armpit.

After a few minutes, the model enters a sauna (hot chamber heated at a temperature of 48° C.) for 10 minutes.

The perspiration is then evaluated by placing on each treated region a sheet of absorbent paper that darkens when it is impregnated with sweat.

The evaluation shows that the absorbent paper is very sparingly impregnated on the left armpit that was treated with compositions (F) and then (A), relative to the right armpit treated with the composition comprising aluminium salts.

### Example 7

A sheet of sulfurized paper is placed in the bottom of a Teflon mould and composition (A) is poured in.

Given that the Teflon mould has an area of about 70 cm², composition (A) is poured in so as to obtain a coating with a thickness of 1 mm.

After drying, after ten days, the sheet of sulfurized paper covered with the coating is delicately removed. Thus, the upper face of the sheet of sulfurized paper is covered with the coating.

The lower face of the sheet of sulfurized paper, which is opposite the face covered with composition (A), is then bonded to a sheet of strong paper. This coating is cut out with a cutter, taking care not to cut the sheet of sulfurized paper. The sheet of strong paper serving as support for the sheet of sulfurized paper makes it possible to maintain cohesion if the cutter notches the sheet of sulfurized paper. The cutting out is performed finely so that the coating is cut into elements of about 2 mm by 2 mm.

A composition containing 3% by weight of active materials of sulfonic polyester sold under the name AQ1350, which is conditioned in a pump-dispenser bottle comprising an ethanol/water mixture (50/50), is sprayed onto these elements. 1 g of this composition is applied to all of the elements. The elements obtained by the film-forming elastomeric polymer of composition (A) are thus covered with a coat formed from an adhesive organic polymer.

The sheet is then applied to the skin at the spot that it is desired to treat. The elements obtained by the film-forming elastomeric polymer of composition (A) adhere satisfactorily to the surface of the skin by means of the adhesive coat. The sheet serving as support and the sheet of sulfurized paper are removed to leave on the skin the elements obtained by the film-forming elastomeric polymer of composition (A).

### Example 8

Formula “G” below is prepared:

| | |
|---|---|
| Pebax 2533 SA 01 (Arkema) | 5% |
| Ethanol | 95% |

The formula is left at 60° C. for 15 days to ensure good dissolution.

After returning to room temperature, it may be applied to the skin or the hair.

The spreading is very easy and the solution dries. No tautness of the skin is felt, and the coating formed is imperceptible to the eye.

After 4 hours, a check may be made to see that the coating is still intact or virtually intact. To do this, one of the edges is lifted up. It is seen that the coating, which is extremely thin (less than 5μ), is virtually intact.

In another test, the following examination is performed:

Bath for 20 minutes, followed by drying. 4 hours later, the same manipulation for checking the integrity of the coating is performed.

The coating is virtually intact.

The same tests were performed with film-forming materials such as Resin 2829-30 or elastomeric resins such as Baycusan C1004 (Bayer).

In the first case, the coating does not withstand either of the two tests.

In the second case, the coating according to the invention withstands relatively well the first of the two tests, but does not withstand the second test. Strong delamination is in particular noted.

Since the coating is slightly non-slip, this aspect can be corrected, if so desired.

In this case, the polymer may be combined with a third compound, for instance a polyamide resin with amine end groups, for instance the product sold under the trade name Versamid 756 (Cognis).

Formula “H” below is prepared:

| | |
|---|---|
| Pebax 2533 SA 01 (Arkema) | 5% |
| Polyamide resin with amine end groups | 10% |
| (Versamid756 from Cognis) | |
| Ethanol | 85% |

Formulation G may be used on the skin in the form obtained. Formulation H is particularly advantageous since it does not have any non-slip effect, it better withstands the inevitable frictions in the region of the armpits.

Agents such as powders, talc, perlite, antimicrobial active agents, antiperspirant active agents such as aluminium or zirconium salts, etc. may also be introduced into the formula. It is also possible to apply formulation G in a first stage, followed by an aqueous formula of an aluminium salt. In the odour tests, it is noted that this process functions just as well as direct application of the aluminium salt to the skin.

It is also possible to apply an antiperspirant composition of aluminium salt in a first stage, and then to apply formulation G or H.

1. Use of a cosmetic composition comprising, in a cosmetically acceptable medium, one or more nonionic, anionic, amphoteric or cationic film-forming elastomeric polymers, which are capable of giving, on drying of the said polymer(s), at room temperature and at a relative humidity of 55%, a material with a mechanical profile defined by at least:
a degree of elongation at break (ε) of greater than or equal to 150%,
an instantaneous recovery (Rᵢ) of greater than or equal to 75% after an elongation of 150%,
a recovery at 300 seconds (R₃₀₀ₛ) of greater than 80%, after an elongation of 150%, for the cosmetic treatment of human perspiration.

2. Use according to claim 1, characterized in that the material has a degree of elongation at break (ε) greater than at least 250%, preferentially ranging from 250% to 1000%, an instantaneous recovery (Rᵢ) ranging from 75% to 100% and a recovery at 300 seconds (R₃₀₀ₛ) ranging from 80% to 100%.

3. Use according to claim 1, characterized in that the film-forming elastomeric polymer(s) are chosen from the group comprising polyurethanes, polyvinyl alcohols, polymers comprising at least one (meth)acrylic unit, and combinations thereof.

4. Use according to claim 1, characterized in that the film-forming elastomeric polymer(s) are polyurethanes that are capable of giving, on drying of the said polyurethane(s), at room temperature and at a relative humidity of 55%, a material, in particular a film, with a degree of elongation at break (ε) of 450%, an instantaneous recovery (Rᵢ) of 88% and a recovery at 300 seconds (R₃₀₀ₛ) of 94%.

5. Use according to claim 1, characterized in that the film-forming elastomeric polymer(s) are polyamide-polyether polymers, in particular copolymers bearing polyamide blocks and polyether blocks and even more particularly copolymers bearing polyamide-6 or polyamide-12 blocks and polyethylene glycol or polytetramethylene glycol blocks.

6. Use according to claim 1, characterized in that the film-forming elastomeric polymer(s) are soluble in an aqueous-alcoholic or aqueous cosmetically acceptable medium or are water-dispersible in an aqueous or aqueous-alcoholic cosmetically acceptable medium.

7. Use according to claim 1, characterized in that the cosmetic composition also comprises one or more pressure-sensitive adhesives.

8. Cosmetic process for treating human perspiration, and optionally the body odours associated with human perspiration, especially underarm odours, characterized in that it consists in applying to the skin a cosmetic composition comprising, in a cosmetically acceptable medium, one or more film-forming elastomeric polymers as defined in any one of claim 1.

9. Cosmetic process for treating human perspiration, and optionally the body odours associated with human perspiration, especially underarm odours, characterized in that it comprises at least the step of applying to the skin a coating formed in situ from a liquid composition comprising at least one polyamide-polyether as defined in claim 5 in solution or in dispersion and at least one organic solvent.

10. Process according to claim 9, characterized in that the following are applied:
a) in a first step, a coating formed in situ from a liquid composition comprising at least one polyamide-polyether as defined in claim 5 in solution or in dispersion and at least one organic solvent,
b) in a second step, a composition (A) comprising, in a cosmetically acceptable medium, at least one agent for treating perspiration; the order of application being irrelevant.

11. Cosmetic assembly comprising at least:
(i) a first composition (A) as defined in claim 10;
(ii) a second composition (B) as defined in claim 10.

12. Cosmetic process for treating human perspiration according to claim 8, characterized in that it comprises a first step consisting in applying to the skin a cosmetic composition A comprising one or more film-forming elastomeric polymers and a second step that consists in mechanically reinforcing the material obtained by the said film-forming elastomeric polymer(s).

13. Process according to claim 8, characterized in that the second step consists in applying a cosmetic composition B comprising one or more anionic polymers chosen from acrylic acid copolymers such as copolymers derived from crotonic acid such as vinyl acetate/vinyl tert-butylbenzoate/crotonic acid terpolymers and crotonic acid/vinyl acetate/vinyl neododecanoate terpolymers, polymers derived from maleic, fumaric or itaconic acids or anhydrides with vinyl esters, vinyl ethers, vinyl halides, phenylvinyl derivatives, acrylic acid and esters thereof, such as the methyl vinyl ether/monoesterified maleic anhydride copolymer.

14. Cosmetic process for treating human perspiration according to claim 8, characterized in that it comprises a first step that consists in applying to the skin a cosmetic composition A comprising one or more film-forming elastomeric polymers and a second step that consists in applying a cosmetic composition B comprising one or more pressure-sensitive adhesives.

15. Cosmetic process for treating human perspiration according to claim 14, characterized in that the pressure-sensitive adhesive(s) are chosen from adhesive organic polymers and particles comprising at least a first solid phase covered over at least part of its surface with one or more adhesive organic polymers.

16. Cosmetic process for treating human perspiration according to claim 8, characterized in that it comprises a first step that consists in applying to the skin a cosmetic composition A comprising one or more film-forming polymers as defined in claim 1 to give an antiperspirant effect, and a second step that consists in reducing the discomfort associated with the elastomeric coating formed on the skin by the deposit of the film-forming elastomeric polymer(s).

17. Process according to claim 16, characterized in that the second step consists in applying a cosmetic composition B that is capable of reducing the discomfort associated with the elastomeric coating formed on the skin by the deposit of the film-forming elastomeric polymer(s) or in physically treating the skin.

18. Process according to claim 16, characterized in that the second step consists in circulating heat on contact with the skin by means of an external source of heat or in irradiating the skin by means of a light source.

19. Process according to claim 17, characterized in that the second step also consists in reinforcing the antiperspirant effect.

20. Process according to claim 16, characterized in that the second step also consists in improving the appearance of the skin.
